SEQUENCE LISTING (6 sequences)

SEQ ID NO: 1 - DNA, 453 bp, Escherichia coli
  source 1..453: mol_type = genomic DNA
atgaaacttttaaaagtagcagcaattgcagcaatcgtattctccggtagcgctctggcaggtgttgttcctcagtacggcggcggcggtaaccacggtggtggcggtaataatagcggcccaaattctgagctgaacatttaccagtacggtggcggtaactctgcacttgctctgcaaactgatgcccgtaactctgacttgactattacccagcatggcggcggtaatggtgcagatgttggtcagggctcagatgacagctcaatcgatctgacccaacgtggcttcggtaacagcgctactcttgatcagtggaacggcaaaaattctgaaatgacggttaaacagttcggtggtggcaacggtgctgcagttgaccagactgcatctaactcctccgtcaacgtgactcaggttggctttggtaacaacgcgaccgctcatcagtac

SEQ ID NO: 2 - DNA, 24 bp, synthetic construct
  source 1..24: mol_type = other DNA
tggagccacccgcagttcgagaaa

SEQ ID NO: 3 - DNA, 48 bp, synthetic construct
  source 1..48: mol_type = other DNA
aaaaaaggcggcagcagccatcaccatcaccatcacagcagcggcggc

SEQ ID NO: 4 - DNA, 12 bp, synthetic construct
  source 1..12: mol_type = other DNA
ggcggcagcagc

SEQ ID NO: 5 - DNA, 555 bp, synthetic construct
  source 1..555: mol_type = other DNA
gctagcaggaggtttgatcctatgaaacttttaaaagtagcagcaattgcagcaatcgtattctccggtagcgctctggcaggtgttgttcctcagtacggcggcggcggtaaccacggtggtggcggtaataatagcggcccaaattctgagctgaacatttaccagtacggtggcggtaactctgcacttgctctgcaaactgatgcccgtaactctgacttgactattacccagcatggcggcggtaatggtgcagatgttggtcagggctcagatgacagctcaatcgatctgacccaacgtggcttcggtaacagcgctactcttgatcagtggaacggcaaaaattctgaaatgacggttaaacagttcggtggtggcaacggtgctgcagttgaccagactgcatctaactcctccgtcaacgtgactcaggttggctttggtaacaacgcgaccgctcatcagtacaaaaaaggcggcagcagccatcaccatcaccatcacagcagcggcggctggagccacccgcagttcgagaaataaaagctt

SEQ ID NO: 6 - DNA, 591 bp, synthetic construct
  source 1..591: mol_type = other DNA
gctagcaggaggtttgatcctatgaaacttttaaaagtagcagcaattgcagcaatcgtattctccggtagcgctctggcaggtgttgttcctcagtacggcggcggcggtaaccacggtggtggcggtaataatagcggcccaaattctgagctgaacatttaccagtacggtggcggtaactctgcacttgctctgcaaactgatgcccgtaactctgacttgactattacccagcatggcggcggtaatggtgcagatgttggtcagggctcagatgacagctcaatcgatctgacccaacgtggcttcggtaacagcgctactcttgatcagtggaacggcaaaaattctgaaatgacggttaaacagttcggtggtggcaacggtgctgcagttgaccagactgcatctaactcctccgtcaacgtgactcaggttggctttggtaacaacgcgaccgctcatcagtacaaaaaaggcggcagcagccatcaccatcaccatcacagcagcggcggctggagccacccgcagttcgagaaaggcggcagcagctggagccacccgcagttcgagaaataaaagctt

(19) United States
(12) Patent Application Publication
(10) Pub. No.: US 2026/0193299 A1
(43) Pub. Date: Jul. 9, 2026

(54) GENE CONSTRUCTS ENCODING FUSION PROTEINS HAVING BINDING CAPACITY FOR STREP-TAG BINDING SUBSTANCES, AND EXPRESSION VECTORS, RECOMBINANT STRAINS AND ANTICANCER COMPOSITIONS COMPRISING THE SAME

(71) Applicant: THE INDUSTRY & ACADEMIC COOPERATION IN CHUNGNAM NATIONAL UNIVERSITY (IAC), Daejeon (KR)

(72) Inventors: Hyo Jin LEE, Daejeon (KR); Heung Jin JEON, Daejeon (KR); Seung Beum SUH, Seoul (KR); Sehoon KIM, Seoul (KR)

(73) Assignee: THE INDUSTRY & ACADEMIC COOPERATION IN CHUNGNAM NATIONAL UNIVERSITY (IAC), Daejeon (KR)

(21) Appl. No.: 19/135,242

(22) PCT Filed: Dec. 5, 2023
(86) PCT No.: PCT/KR2023/019904

§ 371 Date: Jun. 3, 2025

(30) Foreign Application Priority Data
Dec. 6, 2022 (KR) 10-2022-0168743

(51) Int. Cl. C07K 14/245; A61K 35/741; A61K 49/00; C12N 15/74

(52) U.S. Cl. CPC C07K 14/245; A61K 35/741; A61K 49/0097; C12N 15/74; C07K 2319/22

(57) ABSTRACT

A gene construct encoding a fusion protein having specific binding capacity for strep-tag binding substances. The C-terminus of the curli-related CsgA gene of Gram-negative bacteria is linked to a strep-tag gene. The strep-tag binding substances include biotin, avidin, streptavidin, and strep-tactin. The CsgA gene and the strep-tag gene are linked directly or via a linker. A plurality of strep-tag genes are linked in tandem or through a linker.

### CROSS-REFERENCE TO PRIOR APPLICATIONS

This application is a National Stage Patent Application No. PCT/KR2023/019904 (filed on Dec. 5, 2023), which claims priority to Korean Patent Application No. 10-2022-0168743 (filed on Dec. 6, 2022), which are all hereby incorporated by reference in their entirety.

### SEQUENCE LISTING

This application contains a Sequence Listing submitted via XML file and hereby incorporated by reference in its entirety. The Sequence Listing is named “782-0024_Sequence_Listing.xml”, created on May 28, 2025 and 8,126 bytes in size.

### BACKGROUND

The present invention relates to gene constructs encoding fusion proteins having binding capacity for Strep-tag binding substances in a strain targeted to tumor cells, recombinant expression vectors, recombinant strains, and the like comprising the same, wherein the recombinant strains themselves exhibit anti-cancer activity and can be usefully applied to the diagnosis and treatment of tumor cells in combination with anti-cancer agents or contrast agents labeled with a Strep-tag binding substance that specifically binds to Strep-tag, and relates to anti-cancer agents, anti-cancer adjuvants, and tumor imaging aids.

Cancer is a life-threatening disease caused by cells that do not stop proliferating, eventually invade surrounding tissues, and destroy normal cells.

The most effective response system for treating diseases is to strengthen the immune system, but cancer cells are not foreign invaders, so they evade the body's immune response without triggering it. Specific viruses or bacteria infect cancer cells more than normal cells, and the infected bacteria can be targeted by immune cells. Therefore, anti-cancer treatment methods have been proposed to stimulate the body's immune response by deliberately infecting specific viruses or bacteria.

Strains such as Shigella, Vibrio cholera, E. coli, and Bifidobacterium, like Salmonella, have the ability to invade cells in the intestinal tract and target cancer cells. These strains have the advantage of being less pathogenic, so they can be used safely without fear of sepsis. Therefore, research is being conducted to develop strains that can express or suppress substances that help diagnose cancer or aid in anti-cancer treatment, and to use them for anti-cancer therapy.

Leschner et al. (J. Mol. Med. 2010, 88, 763-773) suggested that labeled Salmonella can be effectively used for anti-cancer immunotherapy. A research team at Yale University in the United States announced that genetically modifying Salmonella can maintain its tumor-attacking properties while removing only its toxicity for attenuation, and that injection of the attenuated Salmonella can induce immune stimulation to suppress tumors.

E. coli Nissle (EcN) was discovered by Dr. Alfred Nissle (1874-1965) in 1917, and through over 100 years of safety testing, it has been demonstrated to lack enterotoxins (Shiga toxins, heat-stable and heat-labile toxins), cytotoxins, invasiveness, pathogenic adhesion factors, hemolysins, serum resistance, and antibiotic resistance genes. The non-pathogenicity of this strain has been decoded down to the genetic level, allowing it to be safely utilized for various purposes.

Korean Patent Publication No. 10-2022-0058461 disclosed that a host cell (E. coli, Salmonella) targeting cancer can be utilized for cancer diagnosis by introducing a gene encoding streptavidin. However, in the above patent, when the streptavidin gene alone was introduced, its expression level was low, and when a maltose-binding protein was additionally introduced to increase its expression, its binding to biotin was weakened, so that only a specific gene structure could increase the expression level of streptavidin while maintaining its binding ability to biotin. Additionally, according to this gene construct, it remained in the periplasm of the host cell and was not secreted. However, when streptavidin remains only inside the strain, it binds only to biotin-containing substances absorbed by the strain, which rather reduces its utility, and especially when used as an anti-cancer adjuvant, anti-cancer substances administered externally are targeted to the host cell interior rather than to tumor cells, making it difficult to exert anti-cancer efficacy. Furthermore, the accumulation of expressed streptavidin in the host cell without secretion may affect the survival of the host cell.

Pilus (plural: pili) is involved in bacterial attachment, colonization, and host infection. Salmonella, Shigella, Vibrio cholera, E. coli, and Bifidobacterium mentioned above are Gram-negative bacteria that have curli fibers, a type of pilus, highly aggregated on their outer membrane surface. Curli-related genes include CsgA, CsgB, CsgC, CsgD, CsgE, CsgF, and CsgG, among which CsgA is the main subunit of the curli protein, and it is known that CsgA proteins sequentially bind to CsgB bound to the outer membrane surface to form fibers on the outer membrane surface (see FIG. 1, J Bacteriol, 191 (2): 608-615).

Strep-tag is a peptide consisting of eight amino acids developed to specifically bind to the core of streptavidin, and it exhibits very fast binding kinetics and strong binding affinity with biotin, avidin, streptavidin, and strep-tactin (a modified streptavidin protein with significantly enhanced binding affinity), making it resistant to variations in pH, temperature, and organic solvents, and thus useful for the detection or purification of specific proteins.

### SUMMARY

The object of the present invention is to provide a gene construct that enables stable expression and secretion of a fusion membrane protein having binding capacity for biotin, avidin, streptavidin, strep-tactin, and the like, in order to solve problems of the prior art.

Another object of the present invention is to provide a cancer cell-targeting recombinant strain transformed by said construct and an anticancer composition comprising said recombinant strain.

Another object of the present invention is to provide an anticancer adjuvant or tumor imaging adjuvant that can enhance anticancer efficacy or imaging efficacy by targeting anticancer agents or contrast agents bound to biotin, avidin, streptavidin, strep-tactin, or the like to tumor sites using the recombinant strain.

Another object of the present invention is to provide a method for providing information for cancer diagnosis using said tumor imaging adjuvant.

To achieve the aforementioned objectives, the present invention is characterized by a gene construct encoding a fusion protein having binding capacity for ‘strep-tag binding substances’, wherein the C-terminus of the curli-related CsgA gene (SEQ ID NO: 1) of Gram-negative bacteria is linked to the strep-tag gene (SEQ ID NO: 2). In the present invention, the ‘strep-tag binding substances’ include proteins that specifically bind to strep-tag, such as biotin, avidin, streptavidin, and strep-tactin, without being limited thereto.

In the gene construct of the present invention, the CsgA gene and the strep-tag gene may be directly linked, or may be linked via a linker. In the following embodiments, a construct in which the CsgA gene and the strep-tag gene are linked via a linker with the sequence AAAAAA preceding the gene sequence encoding the amino acid sequence GGSS-HIS6-SSGG (hereinafter simply referred to as ‘HIS’, ‘HIS6’, or ‘H6’; SEQ ID NO: 3) is exemplified (see FIGS. 2 and 3), but the linker is not limited thereto. It is evident that additional linker optimization can be carried out to demonstrate superior expression efficiency, and it will be apparent to those skilled in the art that appropriate modifications to the length or sequence of the linker can be made.

In the gene construct of the present invention, the strep-tag gene may be single or multiple, and in the case of multiple genes, a predetermined linker may be interposed. In the following embodiments, cases are presented where there is one strep-tag gene and where two are connected via a linker (GGCGGCAGCAGC; SEQ ID NO: 4) encoding the amino acid sequence GGSS. The number of strep-tag genes or the length or sequence of the linker may be appropriately modified for optimization.

To improve expression efficiency, a Shine-Dalgarno sequence may be placed upstream of the gene construct of the present invention. It is also evident that appropriate restriction enzyme sites may be placed at the upstream and downstream regions to introduce the gene construct of the present invention into a vector. In the following embodiments, NheI and HindIII sites were inserted at the front and rear of the gene construct for cloning.

The present invention also relates to recombinant expression vectors containing said gene constructs. As used herein, a “vector” is DNA operably linked to express a fusion protein of the invention in a suitable host, which may be a plasmid, a phage particle, or a potential genomic insert. In the specification of the present invention, plasmids and vectors are sometimes used interchangeably. It is evident that the expression vector can be selected and used according to the sequence of the gene construct for high efficiency. In the following embodiments of the invention, the plasmid has an inducible promoter to control the expression of the fusion protein, but it would be possible to utilize a plasmid with a constitutive promoter to ensure that the fusion protein is expressed constitutively, if desired.

In addition, the present invention relates to a recombinant strain that is transformed with the aforementioned recombinant expression vector and expresses the fusion protein on the outer membrane surface. The strain may be harmless to humans, demonstrating immune anti-cancer activity by targeting tumor cells. Examples of such strains include, but are not limited to, E. coli, attenuated Salmonella, Shigella, Acinetobacter, Pseudomonas aeruginosa, Listeria, Clostridium, or gut microbiota (beneficial bacteria), probiotics, and the like. In the following embodiments, harmless E. coli and attenuated Salmonella were used as host strains, among which csgA-his-streptag2-pBad18-asd+/Salmonella aroA aroD asd− was deposited with the Korean Collection for Type Cultures (KCTC) at the Korea Research Institute of Bioscience and Biotechnology on Dec. 5, 2022, and assigned accession number KCTC15224BP.

The present invention also relates to anticancer compositions comprising said recombinant strains as active ingredients. The anticancer recombinant strains of the present invention can be used in anticancer compositions because they themselves target tumor sites and exhibit immune anti-cancer activity.

Furthermore, the present invention relates to an anti-cancer adjuvant comprising the anti-cancer recombinant strain and an anti-cancer agent bound to a strep-tag binding substance.

As described above, the strain of the present invention is targeted to tumor cells, and the fusion protein is expressed (constitutively or inducibly) and is present on the outer membrane surface of the strain. Therefore, when the strain and an anti-cancer agent bound to a strep-tag binding substance are administered together- and if the plasmid is inducible as in the following embodiments, an inducer is also administered at an appropriate time—the anti-cancer agent can be targeted to tumor cells. Thus, the side effects of the anti-cancer agent on other organs can be minimized, anti-cancer activity can be increased, and a lower dose can be used to reduce the burden on patients due to side effects.

An anti-cancer agent may be any nanoparticle, antibody, anti-cancer protein, or small molecule that has little or no intrinsic ability to target tumor cells but exhibits anti-cancer activity, provided that it can be labeled with a strep-tag binding substance that specifically binds to the fusion protein.

Meanwhile, in the present invention, the anti-cancer agent labeled with a strep-tag binding substance binds to the fusion protein, and the release of the anti-cancer agent-through dissociation and targeted delivery to tumor cells—is appropriately regulated, so that it may also function as a sustained-release formulation.

The present invention also relates to a tumor imaging adjuvant comprising the recombinant strain and a contrast agent bound to a strep-tag binding substance. It can be used as a tumor imaging adjuvant that enhances the imaging efficacy of tumor tissue by targeting the contrast agent to the tumor site when co-administered with the recombinant strain and a contrast agent bound to a strep-tag binding substance. One or more contrast agents selected from the group consisting of radionuclides, fluorescent labels, enzyme labels, chemiluminescent markers, gold preparations, and magnetic preparations may be used, and it will be apparent to those skilled in the art to determine the imaging modality based on the type of contrast agent. The tumor imaging adjuvant of the present invention not only enhances the efficiency of tumor imaging but also exhibits anti-cancer activity itself, thus providing more beneficial effects to cancer patients.

The tumor imaging adjuvant of the present invention can be usefully employed for providing information for cancer diagnosis. For example, providing information for cancer diagnosis can be carried out by a method comprising: (A) administering said recombinant strain with tumor-targeting characteristics to a patient suspected of having cancer or undergoing anti-cancer treatment to target tumor cells; (B) inducing expression of said fusion protein if the recombinant strain is inducible; (C) administering to the patient a contrast agent bound to a strep-tag binding substance; and (D) detecting and imaging the contrast agent.

According to the gene construct of the present invention, when introduced into an anti-cancer strain that targets tumor cells and exhibits immune anti-cancer activity, it enables the expression of a fusion protein that specifically binds to a strep-tag binding substance on the outer membrane surface.

Therefore, the anti-cancer recombinant strain into which the gene construct of the present invention is introduced not only exhibits anti-cancer activity by itself but also enables the targeting of anti-cancer agents or contrast agents to tumors, thereby serving as an anti-cancer adjuvant or tumor imaging adjuvant to enhance anti-cancer efficacy and tumor imaging efficacy.

### BRIEF DESCRIPTION OF THE DRAWINGS

FIG. 1 is a schematic gene map of curli fiber components, and a conceptual diagram and photograph showing their expression and the process of fiber formation.

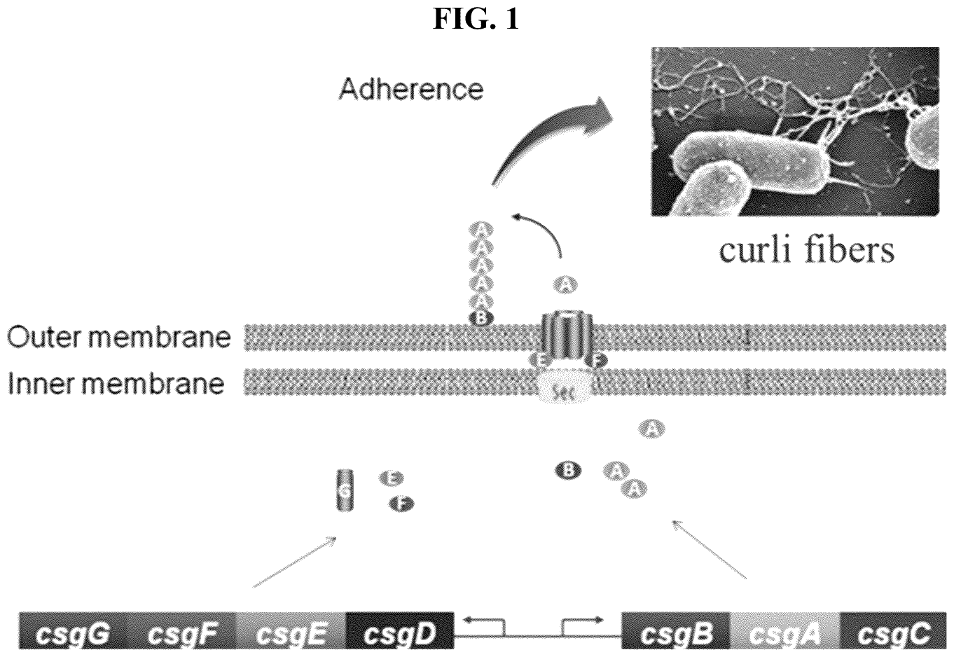

FIG. 2 shows the nucleotide sequence of csgA-HIS-Streptag1 designed in an embodiment of the present invention.

FIG. 3 shows the nucleotide sequence of csgA-HIS-Streptag2 designed in an embodiment of the present invention.

FIG. 4 is an SEM image showing that strep-tag is sufficiently expressed on the surface of an E. coli transformant strain prepared in an embodiment of the present invention.

FIG. 5 is fluorescence showing that strep-tag is sufficiently expressed on the surface of an E. coli transformant strain prepared in an embodiment of the present invention.

FIG. 6 is an SEM image showing that strep-tag is sufficiently expressed on the surface of a Salmonella transformant strain prepared in an embodiment of the present invention.

### DETAILED DESCRIPTION

The present invention will now be described in more detail with reference to the accompanying drawings and examples. However, these drawings and examples are illustrative only to facilitate the explanation of the content and scope of the technical ideas of the invention, and are not intended to limit or change the technical scope of the invention. Based on these examples, it will be apparent to those skilled in the art that various modifications and changes can be made within the scope of the technical ideas of the invention.

### EXAMPLES

### 1. Construction of a Plasmid for Strep-Tag Expression

First, a gene capable of expressing a fusion protein of csgA and strep-tag was designed.

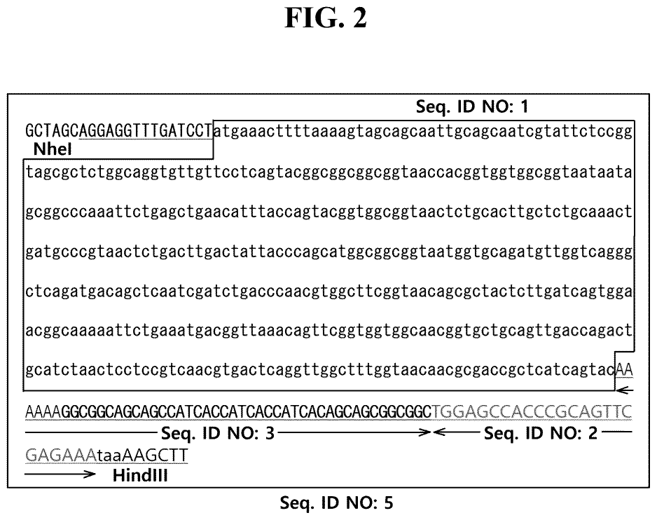

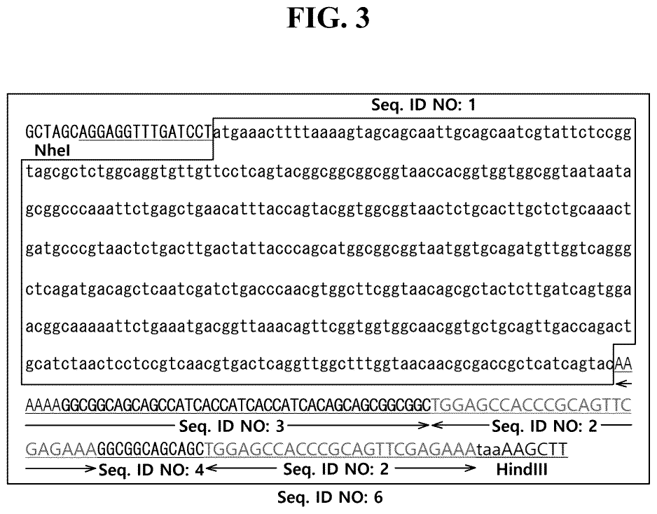

Based on the nucleotide sequence of the csgA gene from E. coli MG1655 K-12 (wild type) (NCBI), two gene constructs were synthesized by inserting one (csgA-HIS-Streptag1) or two (csgA-HIS-Streptag2) strep-tag genes (SEQ ID NO: 2) at the C-terminus of the csgA gene (SEQ ID NO: 1). NheI and HindIII sites were inserted at the 5′ and 3′ ends of the gene constructs for cloning. In addition, to enhance the expression of the csgA gene, a Shine-Dalgarno sequence (AGGAGGTTTGATCCT) was inserted upstream of the csgA structural gene. The gene synthesis was performed by Cosmo Genetech Co., Ltd. The nucleotide sequences of csgA-HIS-Streptag1 and csgA-HIS-Streptag2 (SEQ ID NO: 5 and SEQ ID NO: 6, respectively) designed through the above process are shown in FIG. 2 and FIG. 3, respectively.

The synthesized csgA-his-streptag1 plasmid and csgA-his-streptag2 plasmid were treated with 10 U each of NheI (Takara Co., Ltd.) and SacI (Takara Co., Ltd.) at 37° C. for 2 hours. The reaction products were electrophoresed, and DNA fragments of approximately 550 bp and 590 bp were extracted from each gel using a gel extraction kit (Qiagen) to obtain csgA-his-streptag1: insert (hereinafter referred to as ‘insert 1’) and csgA-his-streptag2 insert (hereinafter referred to as ‘insert 2’).

The pBad18+ asd plasmid (provided by the laboratory of Professor Heon-Man Lim at Chungnam National University) was treated with 10 U each of NheI and SacI enzymes at 37° C. for 2 hours. The reaction products were purified using a PCR purification kit (Qiagen) to obtain vector DNA. The vector DNA was ligated with inserts 1 and 2, respectively, at 25° C. for 30 minutes using a ligation kit (IInvitron Co., Ltd.), and transformed into DH5α competent cells provided by the laboratory of Professor Heon-Man Lim at Chungnam National University. The transformed cells were plated on LB amp solid medium and incubated at 37° C., and six colonies with antibiotic resistance were selected for each ligation.

The selected colonies were treated with 10 U each of NheI (Takara Co., Ltd.) and HindIII (Takara Co., Ltd.) at 37° C. for 1 hour, and the formation of 500-600 bp bands was confirmed by electrophoresis. The nucleotide sequences of the candidates were analyzed (by Cosmo Genetech Co., Ltd.) to complete csgA-his-streptag1-pBad18-asd+ plasmid (hereinafter referred to as ‘plasmid 1’) and csgA-his-streptag2-pBad18-asd+ plasmid (hereinafter referred to as ‘plasmid 2’), wherein the insert 1 and insert 2 genes were correctly inserted, respectively.

### 2. Generation of Transformants Expressing Strep-Tag

(1) E. coli Transformation

Plasmid 1 and plasmid 2 obtained above were transformed into the E. coli Nissle 1917 asd⁻ strain (provided by Professor Heon-Man Lim at Chungnam National University) and plated on LB amp solid medium to select csgA-his-streptag1-pBad18-asd′/E. coli Nissle 1917 asd⁻ and csgA-his-streptag2-pBad18-asd′/E. coli Nissle 1917 asd⁻ colonies, respectively.

### (2) Salmonella Transformation

Except for using the attenuated Salmonella aroA aroD asd⁻ strain (provided by Professor Heon-Man Lim at Chungnam National University) instead of the E. coli Nissle 1917 asd⁻ strain, csgA-his-streptag1-pBad18-asd+/Salmonella aroA aroD asd⁻ and csgA-his-streptag2-pBad18-asd+/Salmonella aroA aroD asd⁻ colonies were selected in the same manner as described in (1) above. Among these, csgA-his-streptag2-pBad18-asd+/Salmonella aroA aroD asd⁻ was deposited with the Korean Collection for Type Cultures (KCTC) at the Korea Research Institute of Bioscience and Biotechnology on Dec. 5, 2022, and assigned accession number KCTC15224BP.

### 3. Verification of Strep-Tag Expression on the Surface of Transformed Strains

The expression of strep-tag on the surface of the transformed strains was confirmed.

(1) Verification of Strep-Tag Expression in E. coli Transformants Through Nanoparticle Binding

Single colonies of the csgA-his-streptag1-pBad18-asd⁺/E. coli Nissle 1917 asd⁻ and csgA-his-streptag2-pBad18-asd⁺/E. coli Nissle 1917 asd⁻ strains prepared in section 2 were each placed in 20 mL of LB amp liquid medium and cultured at 37° C. for 16 hours. After diluting 200 μL of the culture in 20 mL of fresh LB amp liquid medium, the culture was further incubated at 37° C. for approximately 2 hours until the OD600 reached 0.4-0.6. Subsequently, for induction of expression, 200 μL of 20% (w/v) L-arabinose was added to achieve a final concentration of 0.2%, followed by incubation at 37° C. for 6 hours. An equal volume of triple-distilled water was added to the control group. After 6 hours of culture, 2 mL of culture sample with OD600 1.0 was taken and washed three times with motility medium (150 mM NaCl, 2 mM Na₂HPO₄·7H₂O, 1.9 mM KH₂PO₄, 0.1 mM EDTA, 0.01 mM L-methionine, and 10 mM DL-lactate) by centrifugation at 4200 rpm for 5 minutes to prepare 1.0 mL of bacterial sample at OD600 1.0.

Separately, 0.25 mg of avidin-coated iron oxide nanoparticles (200 nm diameter, provided by Professor Jin-Sil Choi of Hanbat National University) were suspended in 1 mL of PBS to prepare a nanoparticle solution.

50 μL of the bacterial sample and 50 μL of the nanoparticle solution prepared above were mixed at 600 rpm using a vortex mixer at room temperature for 30 minutes. After mixing at 600 rpm for 30 minutes using a vortex mixer, the mixture was centrifuged at 4200 rpm for 5 minutes to remove the supernatant. 0.1 mL of 4% glutaraldehyde was added to the pellet and resuspended, then left overnight in a refrigerator for fixation.

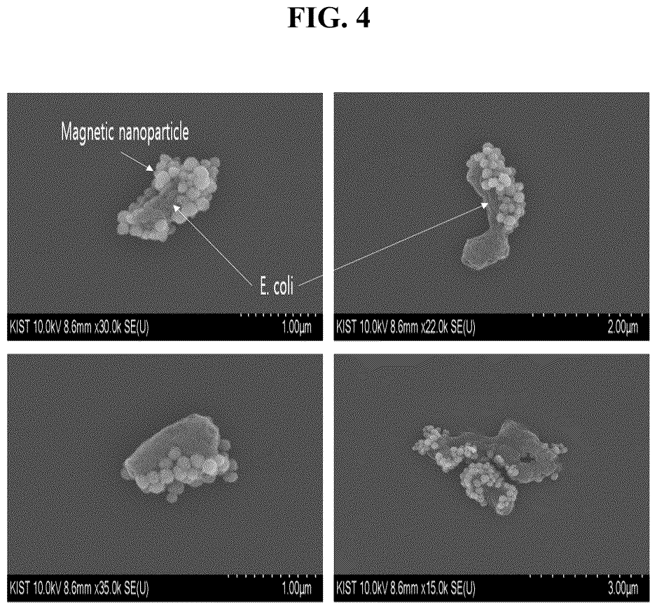

4 μL of the fixed bacterial solution was spotted onto a wafer, dried for 2 hours in a vacuum at 37° C., and then platinum-coated for 20 seconds at 10 mA before observation with FE-SEM (Regulus8230) (FIG. 4). As shown in the figure, the SEM image of the nanoparticle-coated csgA-his-streptag2-pBad18-asd′/E. coli Nissle 1917 asd⁻ strain confirms that nanoparticles are bound to the surface of most bacteria (>85%). This demonstrates that strep-tag is expressed on the pili of the bacterial surface through induction and exerts its binding function. In contrast, the binding of nanoparticles was less than 10% in the csgA-his-streptag-pBad18-asd⁺/E. coli Nissle 1917 asd⁻ strain (not shown), and in the strain without strep-tag activated on the surface (without arabinose induction) (control group), almost no binding with nanoparticles was observed (not shown).

(2) Verification of Strep-Tag Expression in E. coli Transformants by Fluorescence Analysis

To confirm whether the strain activates strep-tag on the bacterial surface (pili), the activation performance of strep-tag was analyzed by staining with DAPI dye (Invitrogen, S11222, streptavidin pacific blue) coated with streptavidin, which binds well with strep-tag.

Bacterial samples of 1.0 mL at OD600 1.0 were prepared in the same manner as described in 3 (1) above.

Separately, a staining solution was prepared by suspending 0.25 mg/mL of streptavidin-coated DAPI dye in PBS.

50 μL of the bacterial sample prepared above and 50 μL of the staining solution were mixed at 600 rpm for 30 minutes at room temperature using a vortex mixer, then centrifuged at 4200 rpm for 5 minutes to remove the supernatant. The final observation sample was prepared by adding 0.1 mL of Motility Medium to the pellet and resuspending it.

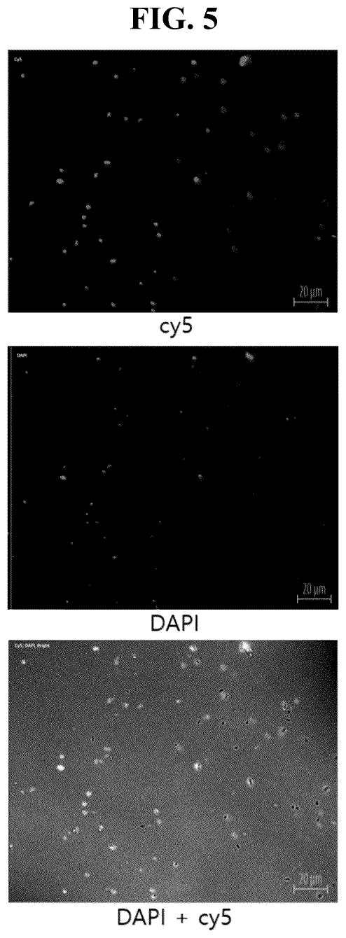

As can be seen from the figure, the positions of most of the bacteria (red, Cy5 fluorescence image) and the DAPI dye (blue, DAPI fluorescence image) coincide, confirming that the strep-tag is activated on the bacterial surface. [If the strep-tag were located inside the bacteria or released externally, the two images would not show the overlapping pattern demonstrated in FIG. 5]

### (3) Verification of Strep-Tag Expression in Salmonella Transformants by Nanoparticle Binding

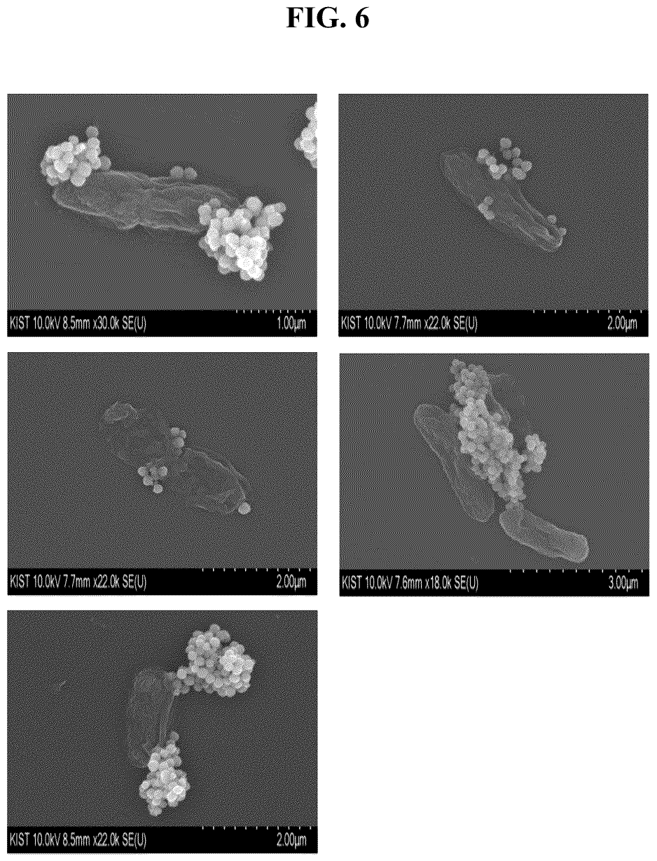

Except for using the csgA-his-streptag2-pBad18-asd+/Salmonella aroA aroD asd-strain instead of the csgA-his-streptag2-pBad18-asd+/E. coli Nissle 1917 asd-strain, the expression of strep-tag in transformed Salmonella was confirmed in the same manner as in 3 (1) above (FIG. 6). As a result, similar to transformed E. coli, nanoparticle binding was present in most (>85%) of the bacteria, indicating that strep-tag is expressed according to the method of the present invention and exerts its binding function in Salmonella as well.

### Appendix to the Specification: Receipt in the Case of an Original Deposit

Name of depositor: The Korean Collection for Type Cultures, Deposit date: Dec. 5, 2022, Accession number: KCTC15224BP, the address of the Korean Collection for Type Cultures is Korea Research Institute of Bioscience and Biotechnology (KRIBB) 181, Ipsin-gil, Jeongeup-si, Jeolllabuk-do, 56212, Republic of Korea. The deposit was made under Budapest Treaty, and that all restrictions imposed by the depository will be irrevocably removed upon the granting of the patent. The deposit is hereby incorporated by reference in its entirety.

1. A gene construct encoding a fusion protein having specific binding capacity for strep-tag binding substances, wherein the C-terminus of the curli-related CsgA gene of Gram-negative bacteria is linked to a strep-tag gene.

2. The gene construct according to claim 1,
wherein the strep-tag binding substances comprise biotin, avidin, streptavidin, and strep-tactin.

3. The gene construct according to claim 1,
wherein the CsgA gene and the strep-tag gene are linked directly or via a linker.

4. The gene construct according to claim 1,
wherein a plurality of strep-tag genes are linked in tandem or through a linker.

5. A recombinant expression vector comprising the gene construct according to claim 1.

6. The recombinant expression vector according to claim 5,
further comprising an inducible promoter operably linked to the gene construct.

7. A recombinant strain transformed with the recombinant expression vector according to claim 5, and expressing a fusion protein on the outer membrane surface.

8. The recombinant strain according to claim 7,
wherein the strain is targeted to tumor cells.

9. The recombinant strain according to claim 7,
wherein the strain is csgA-his-streptag2-pBad18-asd+/Salmonella aroA aroD asd-(accession number KCTC15224BP).

10. An anticancer adjuvant comprising the recombinant strain according to claim 8 and an anticancer agent bound to a strep-tag binding substance.

11. A tumor imaging adjuvant comprising the recombinant strain according to claim 8 and a contrast agent bound to a strep-tag binding substance.

12. A method for providing information for cancer diagnosis or monitoring cancer progression, the method comprising:
(A) administering to a patient suspected of having cancer or undergoing anti-cancer treatment the recombinant strain according to claim 7;
(B) inducing expression of the fusion protein if the recombinant strain is inducible;
(C) administering to the patient a contrast agent bound to a strep-tag binding substance; and
(D) detecting and imaging the contrast agent.

13. A recombinant strain transformed with the recombinant expression vector according to claim 6, and expressing a fusion protein on the outer membrane surface.
